(19) United States
(12) Patent Application Publication
(10) Pub. No.: US 2008/0154049 A1
(43) Pub. Date: Jun. 26, 2008

(54) Method for Acylation of an Aromatic Compound

(75) Inventors: Damien Bourgeois, Clapiers (FR); Joel Turconi, Montpellier (FR); Johann Vastra, Montagny (FR)

Correspondence Address: BUCHANAN, INGERSOLL & ROONEY PC, POST OFFICE BOX 1404, ALEXANDRIA, VA, 22313-1404, US

(73) Assignee: Shasun Pharma Solutions Limited, Northumberland (GB)

(21) Appl. No.: 11/666,877

(22) PCT Filed: Oct. 28, 2005
(86) PCT No.: PCT/FR2005/002716

§ 371 Date: Feb. 14, 2008

(30) Foreign Application Priority Data
Nov. 2, 2004 (FR) 041646

(51) Int. Cl. C07D 307/78

(52) U.S. Cl. 549/462

(57) ABSTRACT

A method for acylation of an aromatic compound, comprising reacting an aromatic compound and an acylating agent of the carboxylic acid type, in the presence of a Lewis acid and of a silylated reagent selected from the group consisting of halosilanes and halosiloxanes.

The present invention relates to a method for acylation of an aromatic compound using an acylating agent, in which method the reagents may comprise reactive functions.

It relates more particularly to reagents comprising a hydroxyl group.

Conventional methods for acylation of aromatic compounds, especially of aromatic hydrocarbons or phenols, use carboxylic acids or carboxylic acid derivatives, such as halide or anhydride, as acylating reagent.

The reaction is generally conducted in the presence of a catalyst of the Lewis acid type (for example AlCl₃, FeCl₃, SnCl₄) or of the Brönsted acid type (trifluoroacetic anhydride, sulfuric acid, hydrofluoric acid, etc.). Reference may be made especially to the work edited by George A. OLAH “Friedel-Crafts and related Reactions”, Volume III, pages 1 to 36 (1964).

The acylation is carried out on substrates that have generally been activated by electron-donor groups and do not contain functions suitable for reacting (for example hydroxyl group) with the acylating agent.

The object of the present invention is to provide a gentle acylation method that is suitable for acylating any type of substrate.

It is, therefore, an object of the invention to be able to carry out the acylation by means of an acylating agent or substrate carrying a hydroxyl group without being necessary to protect the hydroxyl group.

It is an object of the present invention to use a carboxylic acid as the acylating agent, knowing that carboxylic acid is never used as such on account of its poor reactivity but is used either in the form of a derivative, halide or anhydride, or with an added activator, for example Trifluoromethylbenzoic anhydride.

The present invention accomplishes that object and provides a method that enables the above-mentioned disadvantages to be avoided.

A method for acylation of an aromatic compound has now been found, and the present invention relates thereto, that comprises the reaction of an aromatic compound and an acylating agent of the carboxylic acid type, characterised in that the reaction is carried out in the presence of a Lewis acid and of a silylated reagent selected from the halosilanes and halosiloxanes.

The silylated reagent is preferably a polyhalosilane or a polyhalosiloxane.

In the following description of the present invention, “aromatic compound” is understood as meaning the conventional notion of aromaticity as defined in the literature, especially by Jerry MARCH, Advanced Organic Chemistry, 4th Edition, John Wiley and Sons, 1992, pp. 40 and following.

The terms “polyhalosilane” and “polyhalosiloxane” are understood as meaning silicon derivatives carrying at least two halogen atoms, preferably two chlorine atoms, per silicon atom.

More precisely, the present invention provides a method for acylation of an aromatic compound corresponding to the general formula (I):

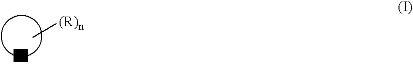

Wherein:
A represents the residue of a ring forming all or part of a monocyclic or polycyclic, aromatic, carbocyclic or heterocyclic system, being possible for said cyclic residue to carry a group R representing a hydrogen atom or one or more identical or different substituents,
n represents the number of substituents on the ring.
The invention is applicable especially to aromatic compounds corresponding to formula (I) wherein A is the residue of an optionally substituted cyclic compound having preferably at least 4, more preferably 5 or 6, atoms in the ring and representing at least one of the following rings:
a monocyclic or polycyclic, aromatic carbocycle,
a monocyclic or polycyclic, aromatic heterocycle comprising at least one of the hetero atoms O, N and S.

Without limiting the scope of the invention, it will be pointed out that the optionally substituted residue A represents the residue:

1. of a monocyclic or polycyclic, aromatic carbocyclic compound.
The term “polycyclic carbocyclic compound” is understood as meaning:
   a compound constituted by at least two aromatic carbocycles and forming between them ortho- or ortho- and peri-condensed systems,
   a compound constituted by at least two carbocycles of which only one is aromatic and which form between them ortho- or ortho- and peri-condensed systems.
      2. of a monocyclic or polycyclic aromatic heterocyclic compound.
The term “polycyclic heterocyclic compound” is defined as:
   a compound constituted by at least 2 heterocycles comprising at least one hetero atom in each ring, of which at least one of the two rings is aromatic, and forming between them ortho- or ortho- and peri-condensed systems,
   a compound constituted by at least one hydrocarbon ring and at least one heterocycle of which at least one of the rings is aromatic, and forming between them ortho- or ortho- and peri-condensed systems.
      3. of a compound constituted by a sequence of rings, as defined in paragraphs 1 and/or 2, bonded together:
by a valence bond,
by an alkylene or alkylidene group having from 1 to 4 carbon atoms, preferably a methylene or isopropylidene group,
by one of the following groups:
—O—, —CO—, —COO—, —OCOO—, —S—, —SO—, —SO₂—, —NR₀—, —CO—NR₀—, in which formulae Rₒ represents a hydrogen atom or an alkyl group having from 1 to 4 carbon atoms, a cyclohexyl group or a phenyl group.

More particularly, the optionally substituted residue A represents the residue:
of an aromatic, carbocyclic monocyclic compound, such as, for example, benzene,
of an aromatic, condensed polycyclic compound, such as, for example, naphthalene,
of an aromatic, carbocyclic, uncondensed polycyclic compound, such as, for example, phenoxybenzene,
of a partially aromatic, carbocyclic, condensed polycyclic compound, such as, for example, tetrahydronaphthalene, 1,2-methylenedioxybenzene,
of a partially aromatic, carbocyclic, uncondensed polycyclic compound, such as, for example, cyclohexylbenzene,
of an aromatic, heterocyclic monocyclic compound, such as, for example, pyridine, furan, thiophene,
of a partially heterocyclic, aromatic, condensed polycyclic compound, such as, for example, quinoline, indole, benzofuran or benzothiophene,
of a partially heterocyclic, aromatic, uncondensed polycyclic compound, such as, for example, a phenylpyridine, a naphthylpyridine,
of a partially heterocyclic, partially aromatic, condensed polycyclic compound, such as, for example, tetrahydroquinoline,
of a partially heterocyclic, partially aromatic, uncondensed polycyclic compound, such as, for example, cyclohexylpyridine.

In the method of the invention, an aromatic compound of formula (I) in which A represents an aromatic, carbocyclic monocyclic compound, such as benzene, or a partially heterocyclic, aromatic bicyclic compound, such as a benzofuran is preferably used.

The aromatic compound of formula (I) may carry one or more substituents.

The number of substituents present on the ring depends on the carbon condensation of the ring and on the presence or absence of unsaturated bonds on the ring.

The maximum number of substituents that may be carried by a ring is readily determined by the person skilled in the art.

In the present text, “more than one” is generally understood as meaning less than 4 substituents on an aromatic ring.

Examples of substituents are given below, but this list is not limiting in nature.

The group or groups R, which may be identical or different, preferably represent(s) one of the following groups:
a hydrogen atom,
a linear or branched alkyl group having from 1 to 6 carbon atoms, preferably from 1 to 4 carbon atoms, such as methyl, ethyl, propyl, isopropyl, butyl, isobutyl, sec-butyl, tert-butyl,
a linear or branched alkenyl group having from 2 to 6 carbon atoms, preferably from 2 to 4 carbon atoms, such as vinyl, allyl,
a linear or branched alkoxy group having from 1 to 6 carbon atoms, preferably from 1 to 4 carbon atoms, such as the groups methoxy, ethoxy, propoxy, isopropoxy, butoxy groups, an alkenyloxy group, preferably an allyloxy group or a phenoxy group,
a cyclohexyl, phenyl or benzyl group,
an acyl group having from 2 to 6 carbon atoms,
a group of the formula:

—R₁—OH

—R₁—COOR₂

—R₁—CHO

—R₁—NO₂

—R₁—CN

—R₁—N(R₂)₂

—R₁—NH—P

—R₁—CO—N(R₂)₂

—R₁—NH—SO₂—R₃

—R₁—X

—R₁—CF₃

in which formulae
R₁ represents a valence bond or a saturated or unsaturated, linear or branched divalent hydrocarbon group having from 1 to 6 carbon atoms, such as, for example, methylene, ethylene, propylene, isopropylene, isopropylidene,
the R₂ groups, which may be identical or different, represent a hydrogen atom or a linear or branched alkyl group having from 1 to 6 carbon atoms,
R₃ represents:
   an alkyl group having from 1 to 10 carbon atoms, preferably from 1 to 4 carbon atoms, and more preferably a methyl or ethyl group, optionally carrying a halogen atom, a CF₃ group or a ammonium N(R₆)₄ group, R₆, which may be identical or different, representing an alkyl group having from 1 to 4 carbon atoms,
   a cycloalkyl group having from 3 to 8 carbon atoms, preferably a cyclohexyl group,
   an aryl group having from 6 to 12 carbon atoms, preferably a phenyl group, optionally carrying an alkyl group having from 1 to 10 carbon atoms, preferably from 1 to 4 carbon atoms, and more preferably a methyl or ethyl group, a halogen atom, a CF₃ group or a NO₂ group,
   a CX₃ group in which X represents a fluorine, chlorine or bromine atom,
   a Cₚ Hₐ F_{b} group in which p represents a number ranging from 1 to 10, b represents a number ranging from 3 to 21 and a+b=2 p+1,
      P represents a protecting group for the amino group,
      X represents a halogen atom, preferably a chlorine, bromine or fluorine atom.

In formula (I), the aromatic ring may carry a —R₁—COOR₂ group in which R₂ represents a hydrogen atom. In the case where the alkylene R₁ group has at least 1 or preferably 2 or 3 carbon atoms, the reaction becomes an intramolecular acylation reaction, because the acylating carboxylic group is carried by the aromatic ring. A cyclic ketone is then obtained.

In formula (I), two R groups located on two adjacent carbon atoms may form, together and with the carbon atoms carrying them, a ring having from 5 to 7 atoms and containing optionally another heteroatom.

When n is greater than or equal to 2, two R groups and the two successive atoms of the aromatic ring may be bonded together by an alkylene, alkenylene or alkenylidene group having from 2 to 4 carbon atoms to form a saturated, unsaturated or aromatic heterocycle having from 5 to 7 carbon atoms. One or more carbon atoms may be replaced by a different hetero atom, preferably oxygen or sulfur. Thus, the R groups may represent a methylenedioxy or ethylenedioxy group or a methylenedithio or ethylenedithio group.

It is possible for the hydrogen atom of the amino group to be blocked by means of a protecting group. Protecting groups conventionally used for such purposes are employed, and special mention may be made of groups like acyl (acetyl, benzoyl), BOC (butyloxycarbonyl), Cbz (carbobenzoxy), Fmoc (9-fluorenylmethoxycarbonyl) or MSOC (methanesulfenyl-2-ethoxycarbonyl). Reference may be made in this connection to the book of Theodora W. Greene et al., Protective Groups in Organic Synthesis, (2nd edition) John Wiley & Sons, Inc.

In formula (I), the group R more particularly represents a —R₁—NH—SO₂—R₃ group in which R₁ represents a valence bond and R₃ represents a methyl, phenyl, tolyl or trifluoromethyl group.

A preferred class of substrates is that which encompasses substrates corresponding to formula (Ia):

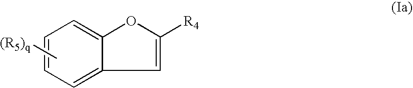

in which formula:
R₄ represents a linear or branched alkyl group having from 1 to 12 carbon atoms, a phenyl group optionally substituted by an alkyl group having from 1 to 4 carbon atoms, or a halophenyl group,
R₅, which may be identical or different, represents a hydrogen atom or a substituent,
q is a number equal to 0, 1, 2 or 3.

In formula (Ia), the benzene ring may carry a substituent.

The invention does not exclude the presence of any type of substituent on the rings, preferably benzene rings, provided that it does not react under the conditions of the invention.

The maximum number of substituents that may be carried by a ring is readily determined by the person skilled in the art.

In formula (Ia), q is a number equal to 0, 1, 2 or 3. Preferably, q is 1.

Examples of substituents are given below, but this list is not limiting in nature.

As examples of preferred groups R₅, there may be mentioned inter alia:
a nitro group,
a linear or branched alkyl group having from 1 to 6 carbon atoms, preferably from 1 to 4 carbon atoms, such as methyl, ethyl, propyl, isopropyl, butyl, isobutyl, sec-butyl, tert-butyl,
an alkoxy group having from 1 to 6 carbon atoms, preferably from 1 to 4 carbon atoms,
a —N(R₂)₂ group, R₂ having the meaning given hereinbefore,
a —NH—P group, P having the meaning given hereinbefore,
a —CO—N(R₂)₂ group, R₂ having the meaning given hereinbefore,
a —NH—SO₂—R₃ group, R₃ having the meaning given hereinbefore,
a halogen atom,
a trifluoromethyl group.

Preferred substrates of the method of the invention correspond to formula (Ia) in which R₅ represents a hydrogen atom, a nitro group, a methyl or ethyl group, a methoxy or ethoxy group.

The substituent is advantageously in the 4-position.

The R₄ group is preferably an alkyl group having from 1 to 4 carbon atoms.

As examples of compounds corresponding to formula (I) there may be mentioned especially:
benzene, toluene, isobutylbenzene, phenol, cresols, anisole, thioanisole, phenetole or veratrole, guaiacol, guetol, hydroquinone, pyrocatechol,
naphthalene, 2-methoxynaphthalene,
phenoxybenzene,
tetrahydronaphthalene, 1,2-methylenedioxybenzene,
cyclohexylbenzene,
pyridine, furan, thiophene,
quinoline, indole, benzofuran or benzothiophene,
the phenylpyridines, the naphthylpyridines,
tetrahydroquinoline,
cyclohexylpyridine.

Preferred compounds of formula (I) are 5-nitro-2-butylbenzofuran, anisole, toluene, monochlorobenzene, xylenes.

With regard to the acylating agent, it may be represented by the following formula:

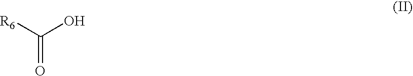

in which formula:
R₆ represents an optionally substituted hydrocarbon group having from 1 to 20 carbon atoms, which may be a linear or branched, saturated or unsaturated acyclic aliphatic group; a monocyclic or polycyclic, saturated, unsaturated or aromatic carbocyclic or heterocyclic group; a linear or branched, saturated or unsaturated aliphatic group carrying a cyclic substituent.

The invention employs more particularly carboxylic acids corresponding to formula (II) in which R₆ represents a linear or branched, saturated, acyclic aliphatic group having preferably from 1 to 12 carbon atoms, more preferably from 1 to 4 carbon atoms.

The invention does not exclude the presence of an unsaturated bond on the hydrocarbon chain, such as one or more double bonds, which may or may not be conjugated, or a triple bond.

The hydrocarbon chain may optionally be interrupted by a hetero atom (for example oxygen or sulfur) or by a functional group, provided that the group does not react, and particular mention may be made of a group such as, especially, —CO—.

The hydrocarbon chain may optionally carry one or more substituents (for example halogen, ester), provided that they do not interfere with the acylation reaction. Accordingly, R₆ may represent a perfluorinated chain of the formula:

—[CF₂]_{w}—CF₃

in which formula w represents a number ranging from 0 to 10.

The linear or branched, saturated or unsaturated, acyclic aliphatic group can optionally carry a cyclic substituent. The term “ring” is understood as meaning a saturated, unsaturated or aromatic carbocyclic or heterocyclic ring.

The acyclic aliphatic group may be bound to the ring by a valence bond, a hetero atom or a functional group such as oxy, carbonyl, carboxy, sulfonyl, etc.

As examples of cyclic substituents, it may be cited cycloaliphatic, aromatic or heterocyclic substituents, especially cycloaliphatic substituents comprising 6 carbon atoms in the ring or benzene substituents, those cyclic substituents themselves optionally carrying any substituent, provided that they do not interfere with the reactions that occur in the method of the invention. Particular mention may be made of alkyl and alkoxy groups having from 1 to 4 carbon atoms.

Among the aliphatic groups carrying a cyclic substituent, more particular mention may be made of cycloalkylalkyl groups, for example cyclohexylalkyl, or aralkyl groups having from 7 to 12 carbon atoms, especially benzyl or phenylethyl.

In formula (II), R₆ may also represent a saturated or unsaturated carbocyclic group having preferably 5 or 6 carbon atoms in the ring; a saturated or unsaturated heterocyclic group having especially 5 or 6 atoms in the ring, including 1 or 2 hetero atoms such as nitrogen, sulfur and oxygen atoms, a condensed or uncondensed, aromatic carbocyclic or heterocyclic monocyclic group, preferably phenyl, pyridyl, pyrazolyl, imidazolyl, or a polycyclic (preferably bicyclic), preferably naphthyl.

When the group R₆ contains a ring, the ring can likewise carry a substituent, represented by R₇. The substituent can be of any kind, provided that it does not interfere with the principal reaction. The number of substituents is generally not more than 4 per ring, more often than not 1 or 2.

The following may be mentioned especially as more particular examples of substituents:
a linear or branched alkyl group having from 1 to 6 carbon atoms, preferably from 1 to 4 carbon atoms, such as methyl, ethyl, propyl, isopropyl, butyl, isobutyl, sec-butyl, tert-butyl,
a linear or branched alkenyl group having from 2 to 6 carbon atoms, preferably from 2 to 4 carbon atoms, such as vinyl, allyl,
a linear or branched alkoxy group having from 1 to 6 carbon atoms, preferably from 1 to 4 carbon atoms, such as the groups methoxy, ethoxy, propoxy, isopropoxy, butoxy, a 2-oxypropionic group,
a —OH group,
a —CHO group,
an acyl group having from 2 to 6 carbon atoms,
a —COOR₂ group, wherein R₂ has the meaning given hereinbefore,
a —NO₂ group,
a CO—N(R₂)₂ group, wherein R₂ has the meaning given hereinbefore,
a halogen atom, preferably a fluorine, chlorine, bromine atom,
a —CF₃ group.

Among all the meanings given hereinbefore for R₆, it preferably represents a linear or branched alkyl group having from 1 to 12 carbon atoms, preferably from 1 to 6 carbon atoms, or a phenyl group.

As carboxylic acids corresponding to formula (II) particularly the following carboxylic acids are used more particularly:
saturated aliphatic monocarboxylic acids such as formic acid, acetic acid, propionic acid, butyric acid, isobutyric acid, valeric acid, isovaleric acid, pivalic acid, lauric acid, myristic acid, palmitic acid, stearic acid,
saturated aliphatic dicarboxylic acids such as oxalic acid, malonic acid, succinic acid, glutaric acid, adipic acid, pimelic acid, suberic acid, azelaic acid, sebacic acid,
unsaturated aliphatic monocarboxylic or dicarboxylic acids such as crotonic acid, isocrotonic acid, oleic acid, maleic acid, fumaric acid, citraconic acid, mesaconic acid,
saturated or unsaturated carbocyclic carboxylic acids such as camphoric acid, chrysanthemic acid,
heterocyclic carboxylic acids such as furancarboxylic acids, thiophenecarboxylic acids, pyrrolecarboxylic acids, pyrazinecarboxylic acids, nicotinic acid, isonicotinic acid, picolinic acid,
aromatic carbocyclic carboxylic acids such as benzoic acid, phthalic acid, isophthalic acid, terephthalic acid, naphthalenecarboxylic acids, toluic acids,
saturated arylaliphatic carboxylic acids such as, especially arylpropionic acids such as 2-phenylpropionic acid, 2-[4-(2-butyl)phenyl]propionic acid, (3-benzoyl-2-phenyl)propionic acid, 2-(6-methoxy-2-naphthyl)propionic acid, or unsaturated acids such as, for example 2-phenylpropenoic acid, cinnamic acid,
halogenated carboxylic acids such as monochloroacetic acid, dichloroacetic acid, trichloroacetic acid, monochloropropionic acid, α-bromopropionic acid, α-bromobutyric acid, trifluoroacetic acid,
aliphatic, cycloaliphatic, arylaliphatic hydroxy acids, such as glycolic acid, lactic acid, glyceric acid, 2-hydroxybutanoic acid, 3-hydroxybutanoic acid, 2-methyllacetic acid, 2-hydroxy-4-methylthiobutanoic acid, tartronic acid, malic acid, tartaric acid, 1-hydroxycyclopropanecarboxylic acid, 2-hydroxyphenyl-propanoic acid, 2-hydroxycinnamic acid, 3-hydroxycinnamic acid, 4-hydroxy-cinnamic acid,
the following hydroxybenzoic acids: 2-hydroxybenzoic acid (salicylic acid), 3-hydroxybenzoic acid, 4-hydroxybenzoic acid, 3-methylsalicylic acid, 4-methyl-salicylic acid, 5-methylsalicylic acid, 3-hydroxy-4-methylbenzoic acid, 3-methoxysalicylic acid, 4-methoxysalicylic acid, 5-methoxysalicylic acid, 3-hydroxy-4-methoxybenzoic acid (isovanillic acid), 4-hydroxy-3-methoxy-benzoic acid (vanillic acid), 3-hydroxy-4,5-dimethoxybenzoic acid, 4-hydroxy-3,5-dimethoxybenzoic acid (syringic acid), 5-hydroxyisophthalic acid, 3-aminosalicylic acid, 4-aminosalicylic acid, 5-aminosalicylic acid, 3-hydroxy-2-aminobenzoic acid, 3-nitrosalicylic acid, 3-hydroxy-4-nitrobenzoic acid, 4-hydroxy-3-nitrobenzoic acid, 3-hydroxy-4-methyl-2-nitrobenzoic acid, 3,5-diiodosalicylic acid, 2,3-dihydroxybenzoic acid, 2,4-dihydroxybenzoic acid, 2,5-dihydroxybenzoic acid, 2,6-dihydroxybenzoic acid, 3,4-dihydroxybenzoic acid (protocatechic acid), 3,5-dihydroxybenzoic acid, 3,5-dihydroxy-4-methylbenzoic acid, 2,3,4-trihydroxybenzoic acid, 2,4,6-trihydroxybenzoic acid, 3,4,5-trihydroxybenzoic acid,
alkoxy and phenoxy acids such as methoxyacetic acid, phenoxyacetic acid, 2,4-dichlorophenoxyacetic acid, phenoxypropionic acid, 2,4-dichlorophenoxy-propionic acid, p-hydroxyphenoxypropionic acid, m-chlorophenoxypropionic acid, 4-phenoxybenzoic acid, (4-carboxy-4-phenoxy)benzoic acid, piperonylic acid,
oxo acids such as 2-acetylbenzoic acid, 4-acetylbenzoic acid, 2-benzoyl-benzoic acid, 4-benzoylbenzoic acid,
acyloxy acids such as 3-benzoyloxypropionic acid, 2-acetoxybenzoic acid, 4-acetoxybenzoic acid,
amino acids such as 2-acetamidoacrylic acid, 2-acetamidobenzoic acid, 3-acetamidobenzoic acid, 4-acetamidobenzoic acid.

The carboxylic acids that are preferably used are acetic acid, propionic acid, isobutyric acid, lactic acid, trifluoroacetic acid, phenylacetic acid, 3-phenyl-propionic acid, the benzoic acids and the hydroxybenzoic acids mentioned later.

With regard to the acylating reagent, it corresponds more particularly to formula (IIa):

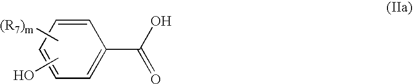

in which:
R₇, which may be identical or different, represents a hydrogen atom or a substituent,
m is a number less than 4.

With regard to the R₇ substituents, they may be of any kind. Examples have been given hereinbefore within the scope of the definition of the substituents relating to the general formula (II).

Among the above-mentioned groups, R₇ is more particularly a hydrogen atom, an alkyl or alkoxy group having preferably from 1 to 4 carbon atoms, or a nitro group or a nitrile group.

In formula (IIa), m is preferably 0 or 1.

As examples of acylating reagents corresponding to formula (IIa), benzoic acid, 2-hydroxybenzoic acid, 3-hydroxybenzoic acid, 4-hydroxybenzoic acid, para-anisic acid, meta-anisic acid, ortho-anisic acid, para-toluenic acid, the chlorobenzoic acids, preferably 4-chlorobenzoic acid, trifluoroparatoluenic acid may be more particularly mentioned.

It is also possible to use salts of carboxylic acids, such as, for example, the sodium or potassium salts.

According to the method of the invention, the acylation of the compound (I) is carried out by means of the compound of formula (II), in the presence of a Lewis acid.

In the present application, “Lewis acid” is understood as meaning, in accordance with the common definition, compounds that are duplet acceptors.

It is possible to use especially the Lewis acids mentioned in the book edited by George A. OLAH “Friedel-Crafts and related Reactions”, Volume I, pages 191 to 197 (1963).

It is possible to use mineral or organic Lewis acids.

As examples of salts containing an organic counter-ion, especially the acetate, propionate, benzoate, methanesulfonate, trifluoromethanesulfonate of the metal or metalloid elements of groups (IIIa), (IVa), (VIII), (IIb), (IIIb), (IVb), (Vb) and (VIb) of the periodic table of elements may be mentioned.

With regard to the salts comprising an inorganic counter-ion, the chlorides, bromides, iodides, sulfates, phosphates, nitrates, oxides and analogous products of the metal or metalloid elements of groups (IIa), (IIIa), (IVa), (VIII), (IIb), (IIIb), (IVb), (Vb) and (Vlb) of the periodic table of elements may be mentioned.

In the present application, reference is made hereinbelow to the periodic table of elements published in Bulletin de la Société Chimique de France, No. 1 (1966).

The salts employed in the method of the invention are more particularly those of the elements of group (IIa) of the periodic table preferably magnesium; of group (IIIa) preferably scandium, yttrium and the lanthanides; of group (IVa) preferably titanium, zirconium; of group (VIII) preferably iron; of group (IIb) preferably zinc; of group (IIIb) preferably boron, aluminium, gallium, indium; of group (IVb) preferably tin; of group (Vb) preferably bismuth; of group (Vlb) preferably tellurium.

Among the inorganic salts, the metal halides and preferably magnesium chloride MgCl₂, zirconium chloride ZrCl₄, ferric chloride FeCl₃, zinc chloride ZnCl₂, aluminium chloride AlCl₃, aluminium bromide AlBr₃, gallium chloride GaCl₃, indium chloride InCl₃, stannic chloride SnCl₄, bismuth chloride BiCl₃, boron trifluoride BF₃ may be mentioned.

With regard to the organic salts, the rare earth and/or bismuth salts of trifluoromethanesulfonic acid, commonly called “triflic acid”, are preferably employed.

“Rare earth” is understood as meaning the lanthanides having an atomic number of from 57 to 71, and yttrium as well as scandium.

In the method of the invention there come into consideration more particularly the following rare earths: lanthanum, ytterbium, lutecium and/or scandium.

Rare earth triflates are known products which are described in the literature, especially in US-A-3 615 169. They are generally obtained by reaction of the rare earth oxide and trifluoromethanesulfonic acid.

The bismuth salts of trifluoromethanesulfonic acid, known as “triflic acid”, described in patent application PCT/FR96/01488 may likewise be used in the method of the invention.

Among the various Lewis acids mentioned above, it is preferred to use the iron(III) halides, preferably ferric chloride.

It is likewise possible to use a mixture of Lewis acids.

A silylated reagent selected from the halosilanes and halosiloxanes is likewise used as reagent in the method of the invention.

Halosilanes may be represented by the following formula:

(R₈)_{z}—Si—X_{4-z} (IIIa)

in which:
R₈ represents a hydrogen atom, an alkyl, alkenyl, cycloalkyl, aryl or arylalkyl group,
R₈ represents at most one hydrogen atom,
X represents a chlorine, bromine or iodine atom, preferably a chlorine atom,
z is a number from 0 to 2, preferably equal to 1 or 2.

Halosiloxanes may be represented by the following formula:

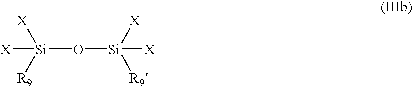

in which:
R₉ and R₉′, which may be identical or different, represent an alkyl, alkenyl, cycloalkyl, aryl or arylalkyl group,
X represents a chlorine, bromine or iodine atom, preferably a chlorine atom.

In formulae (IIIa) and (IIIb), “alkyl” is understood as meaning a linear or branched hydrocarbon chain having from 1 to 10 carbon atoms and preferably from 1 to 4 carbon atoms.

Examples of preferred alkyl groups are especially methyl, ethyl, propyl, isopropyl, butyl, isobutyl, tert-butyl.

“Alkenyl” is understood as being a linear or branched hydrocarbon group having from 2 to 6 carbon atoms, preferably a vinyl group.

“Cycloalkyl” is understood as being a monocyclic cyclic hydrocarbon group comprising 5 or 6 carbon atoms, preferably a cyclopentyl or cyclohexyl group.

“Aryl” is understood as being an aromatic mono- or poly-cyclic, preferably mono- or bi-cyclic, group comprising from 6 to 12 carbon atoms, preferably phenyl.

“Arylalkyl” is understood as being a linear or branched hydrocarbon group carrying a monocyclic aromatic ring and comprising from 7 to 12 carbon atoms, preferably benzyl.

The preferred halosilane corresponds to formula (IIIa) in which the group R₈ is an alkyl group having from 1 to 4 carbon atoms or a phenyl group; the group R₈ preferably being a methyl group.

It should be noted that in formula (IIIa) the R₈ group may be a hydrocarbon group of any kind. However, that group does not occur in the final product. Consequently, it is valuable from an economic point of view for that reagent to be as simple as possible. Accordingly, the group R₈ is preferably a methyl group, although it may have a meaning other than that mentioned above.

Likewise, the invention does not exclude the use of derivatives comprising more than one silicon atom, for example two silicon atoms. An example which may be mentioned is 1,1,2,2-tetrachloro-1,2-dimethylsilane. However, such derivatives are not preferred for economic reasons.

As examples of halosilanes that are preferably used, halosilanes such as Me₂SiCl₂, MeSiCl₃, SiCl₄, MeSiHCl₂ may be mentioned.

An alkyl- and/or a phenyl-halosilane, more preferably an alkylhalosilane is (are) preferably chosen.

Preferred halosilanes are: Me₂SiCl₂, MeSiCl₃.

The preferred halosiloxane corresponds to formula (IIIb) in which R₉ and R₉′ are identical and represent a linear or branched alkyl group having from 1 to 4 carbon atoms, or a vinyl group.

More specific examples which may be mentioned include especially 1,1,3,3-tetrachloro-1,3-dimethylsiloxane, 1,1,3,3-tetrachloro-1,3-diethylsiloxane, 1,1,3,3-tetrachloro-1,3-diisopropylsiloxane, 1,1,3,3-tetrachloro-1,3-divinyl-siloxane.

According to a variant of the invention, it is possible to produce the halosiloxane in situ according to techniques known to the person skilled in the art.

The silylated reagent that is preferably chosen in the method of the invention is a halosilane, preferably a polyhalosilane.

According to the invention, the acylation reaction is advantageously conducted in a liquid phase comprising the aromatic compound and the acylating agent, in the presence of a Lewis acid and of a silylated reagent.

There is generally used an organic solvent that has been less activated than the starting substrate and that preferably is a solvent therefore.

It is preferred to use a polar or non-polar aprotic solvent.

Examples of solvents according to the present invention which may be mentioned include in particular halogenated or non-halogenated aliphatic or aromatic hydrocarbons.

As examples of aliphatic hydrocarbons, the paraffins such as, especially, hexane and cyclohexane, aromatic hydrocarbons and, more particularly, aromatic hydrocarbons such as, especially, benzene, toluene, xylenes, cumene, petroleum fractions constituted by a mixture of alkylbenzenes, especially fractions of the Solvesso® type may be mentioned more particularly.

With regard to the aliphatic or aromatic, halogenated hydrocarbons, perchlorinated hydrocarbons such as, especially, tetrachloroethylene, hexachloroethane; partially chlorinated hydrocarbons such as dichloromethane, 1,2-dichloroethane, monochlorobenzene, dichlorobenzene may be mentioned more particularly.

The preferred solvent is monochlorobenzene.

It is also possible to use a mixture of organic solvents.

It is possible to use the starting substrate as the reaction solvent.

As mentioned above, the aromatic compound is reacted with an acylating agent, optionally in the presence of a reaction solvent as defined and in the presence of a Lewis acid and of a silylated reagent.

The ratio between the number of moles of acylating agent and the number of moles of aromatic compound may vary, for example, between 0.5 and 1.5 and is preferably between 0.8 and 1.2.

The amount of Lewis acid, expressed by the ratio between the number of moles of catalyst and the number of moles of compound of formula (I), varies more often between 0.01 and 2, preferably between 0.2 and 1.5.

By way of example, said ratio is advantageously chosen to be equal to at least 1 for FeCl₃ and in the region of 0.1 for bismuth triflate.

The amount of silylated reagent is expressed relative to the number of halogen atoms. Accordingly, the ratio between the number of halogen atoms and the number of moles of compound of formula (II) varies between 1 and 10 and preferably between 2 and 6.

When a solvent is used, the amount of organic solvent is generally chosen so that the concentration by weight of the compound of formula (I) in the solvent varies between 5 and 80%, preferably between 15 and 40%.

The temperature at which the acylation reaction is carried out depends on the reactivity of the starting substrate and of the acylating agent.

It is between 0° C. and 160° C., preferably between 20° C. and 100° C. and more preferably between 30° C. and 60° C.

The reaction is generally conducted at atmospheric pressure, but lower or higher pressures may also be suitable. The reaction is carried out under autogenous pressure when the reaction temperature is higher than the boiling temperature of the reagents and/or of the products.

It is preferred to conduct the reaction under a controlled atmosphere of inert gases such as nitrogen or noble gases, for example argon.

The method may be carried out discontinuously or continuously.

From a practical point of view, the method is simple to carry out. The various reagents are mixed and the mixture is heated.

A preferred form consists in gradually adding one of the reagents to the reaction medium comprising the others.

It is possible to add the aromatic compound to the medium comprising the acylating agent, the Lewis acid and the silylated reagent.

It is likewise possible to add the aromatic compound and the silylated reagent to a medium comprising the acylating agent and the Lewis acid.

It is likewise possible gradually to add the Lewis acid to the medium comprising the other reagents.

After the reagents have been brought into contact, the reaction mixture is maintained at the desired temperature.

The reaction time is dependent on many parameters. More often, it is from 1 hour to 6 hours.

At the end of the reaction, a compound of the protic type, for example an alcohol (methanol, ethanol, isopropanol) and/or water, is added and an organic phase comprising the acylated aromatic compound is recovered, which acylated aromatic compound may be recovered in the conventional manner, by distillation or by recrystallisation from a suitable solvent, for example the solvent of the reaction.

There is obtained a product corresponding to the general formula:

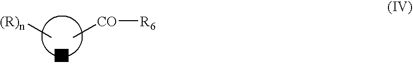

in which formula A, R, R₆ and n have the meaning given hereinbefore.

The preferred product obtained corresponds more particularly to the following formula (IVa):

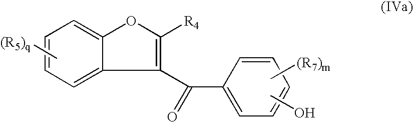

in which formula R₄, R₅, R₇, q and m have the meaning given hereinbefore.

The method of the invention has many advantages. It comprises only a single step. The hydroxyl group is not protected. Accordingly, contrary to the prior art, where the hydroxyl group is protected in the form of a methoxy or ethoxy group, deprotection does not result in the formation of an alkyl halide, a product that is to be avoided because it is toxic. In addition, there is less saline effluent to be treated.

The Examples which follow illustrate the invention.

The abbreviations used in the examples have the meanings indicated below.
NBB: 2-butyl-5-nitrobenzofuran
HNBB: 2-butyl-3-(4-hydroxybenzoyl)-5-nitrobenzofuran
RC (Y): rate of conversion of the substrate Y, corresponding to the ratio of the number of moles of Y converted to the original number of moles of Y
AY (X): actual yield of the compound X, corresponding to the ratio of the number of moles of X formed to the maximum theoretical number of moles of X
YC (X): selectivity of the compound X, corresponding to the ratio AY (X) to RC (Y)
HPLC: high-performance liquid chromatography
eq.: equivalent
m.p.: melting point.

### EXAMPLE 1

5.53 g of 4-hydroxybenzoic acid, 25 ml of monochlorobenzene, 8.37 g of methyltrichlorosilane, 8.77 g of 5-nitro-2-butylbenzofuran and 6.49 g of iron(III) chloride are introduced successively at 23° C., under an inert atmosphere, into a 100 ml reactor equipped with a mechanical stirring system, a cooling apparatus and a temperature probe.

The mixture is then heated to 40° C., with stirring, over a period of 5 hours, and then cooled to 20° C.

8 ml of absolute ethanol are then added in 10 minutes, and then stirring is carried out for a further 20 minutes at 20° C.

The totality of the reaction mass is then weighed, and its composition is determined by HPLC.

The following results are obtained:
RC (NBB)=95%
YC(HNBB)=78%.

### EXAMPLE 2

6.63 g of 4-hydroxybenzoic acid, 25 ml of monochlorobenzene, 8.37 g of methyltrichlorosilane, 8.77 g of 5-nitro-2-butylbenzofuran and 6.49 g of iron(III) chloride are introduced successively at 23° C., under an inert atmosphere, into a 100 ml reactor equipped with a mechanical stirring system, a cooling apparatus and a temperature probe.

The mixture is then heated to 40° C., with stirring, over a period of 5 hours, and then cooled to 20° C.

8 ml of absolute ethanol are then added in 10 minutes, and then stirring is carried out for a further 20 minutes at 20° C.

The totality of the reaction mass is then weighed, and its composition is determined by HPLC.

The following results are obtained:
RC (NBB)=93%
YC (HNBB)=81%.

### EXAMPLE 3

5.53 g of 4-hydroxybenzoic acid, 25 ml of monochlorobenzene, 8.37 g of methyltrichlorosilane, 8.77 g of 5-nitro-2-butylbenzofuran and 7.78 g of iron(III) chloride are introduced successively at 23° C., under an inert atmosphere, into a 100 ml reactor equipped with a mechanical stirring system, a cooling apparatus and a temperature probe.

The mixture is then heated to 40° C., with stirring, over a period of 5 hours, and then cooled to 20° C.

8 ml of absolute ethanol are then added in 10 minutes, and then stirring is carried out for a further 20 minutes at 20° C.

The totality of the reaction mass is then weighed, and its composition is determined by HPLC.

The following results are obtained:
RC (NBB)=100%
YC(HNBB)=76%.

### EXAMPLE 4

5.53 g of 4-hydroxybenzoic acid, 25 ml of monochlorobenzene, 5.98 g of methyltrichlorosilane, 8.77 g of 5-nitro-2-butylbenzofuran and 6.49 g of iron(III) chloride are introduced successively at 23° C., under an inert atmosphere, into a 100 ml reactor equipped with a mechanical stirring system, a cooling apparatus and a temperature probe.

The mixture is then heated to 40° C., with stirring, over a period of 5 hours, and then cooled to 20° C.

8 ml of absolute ethanol are then added in 10 minutes, and then stirring is carried out for a further 20 minutes at 20° C.

The totality of the reaction mass is then weighed, and its composition is determined by HPLC.

The following results are obtained:
RC (NBB)=93%
YC(HNBB)=77%.

### EXAMPLE 5

5.53 g of 4-hydroxybenzoic acid, 25 ml of monochlorobenzene, 8.37 g of methyltrichlorosilane and 6.49 g of iron(III) chloride are introduced successively at 23° C., under an inert atmosphere, into a 100 ml reactor equipped with a mechanical stirring system, a cooling apparatus and a temperature probe.

The mixture is then heated to 40° C., with stirring, and 8.77 g of 5-nitro-2-butylbenzofuran are poured in 1 hour.

The temperature of the reaction medium is then maintained at 40° C. for 4 hours, and then cooling to 20° C. is carried out.

8 ml of absolute ethanol are then added in 10 minutes, and then stirring is carried out for a further 20 minutes at 20° C.

The totality of the reaction mass is then weighed, and its composition is determined by HPLC.

The following results are obtained:
RC (NBB)=88%
YC (HNBB)=79%.

### EXAMPLE 6

5.53 g of 4-hydroxybenzoic acid, 25 ml of monochlorobenzene, 8.37 g of methyltrichlorosilane and 8.77 g of 5-nitro-2-butylbenzofuran are introduced successively at 23° C., under an inert atmosphere, into a 100 ml reactor equipped with a mechanical stirring system, a cooling apparatus and a temperature probe.

The mixture is then heated to 40° C., with stirring, and 6.49 g of iron(III) chloride are added in small portions in 45 minutes.

The temperature of the reaction medium is then maintained at 40° C. for 5 hours, and then cooling to 20° C. is carried out.

8 ml of absolute ethanol are then added in 10 minutes, and then stirring is carried out for a further 20 minutes at 20° C.

The totality of the reaction mass is then weighed, and its composition is determined by HPLC.

The following results are obtained:
RC (NBB)=95%
YC (HNBB)=78%.

### EXAMPLE 7

690 mg of 4-hydroxybenzoic acid, 3 ml of monochlorobenzene, 1.11 g of tetra-chlorosilane, 1.12 g of 5-nitro-2-butylbenzofuran and 950 mg of iron(III) chloride are introduced successively at 23° C., under an inert atmosphere, into a 30 ml reactor equipped with a magnetic stirring system.

The mixture is then heated to 50° C., with stirring, over a period of 5 hours, and then cooled to 20° C.

1 ml of absolute ethanol is then added.

The following results are obtained:
AY (HNBB)=75%.

### EXAMPLE 8

690 mg of 4-hydroxybenzoic acid, 3 ml of monochlorobenzene, 1.33 g of dimethyldichlorosilane, 1.12 g of 5-nitro-2-butylbenzofuran and 820 mg of iron(II) chloride are introduced successively at 23° C., under an inert atmosphere, into a 30 ml reactor equipped with a magnetic stirring system. The mixture is then heated to 50° C., with stirring, over a period of 5 hours, and then cooled to 20° C.

1 ml of absolute ethanol is then added.

The following results are obtained:
AY (HNBB)=76%.

### EXAMPLE 9

690 mg of 4-hydroxybenzoic acid, 3 ml of monochlorobenzene, 1.04 g of methyltrichlorosilane, 1.12 g of 5-nitro-2-butylbenzofuran and 330 mg of bismuth(III) triflate are introduced successively at 23° C., under an inert atmosphere, into a 30 ml reactor equipped with a magnetic stirring system. The mixture is then heated to 60° C., with stirring, over a period of 15 hours, and then cooled to 20° C.

1 ml of absolute ethanol is then added.

The following results are obtained:
AY (HNBB)=10%.

### EXAMPLE 10

8.71 g of 4-hydroxybenzoic acid and 41 g of monochlorobenzene are introduced successively, under an inert atmosphere, into a 250 ml reactor equipped with a mechanical stirring system, a cooling apparatus and a temperature probe.

The mixture is then heated to 40° C., with stirring, and 13.45 g of methyltrichloro-silane and 12.65 g of iron(III) chloride are added thereto successively, and stirring is then carried out for 15 minutes at 40° C.

A mixture of 13.16 g of 5-nitro-2-butylbenzofuran and 6.3 g of monochloro-benzene is then poured in 12 minutes.

The temperature of the reaction medium is then maintained at 40° C. for 3 hours, and then cooling to 30° C. is carried out.

12 g of absolute ethanol are then added in 17 minutes, and stirring is then carried out for a further 10 minutes at 30° C.

The totality of the reaction mass is then weighed, and its composition is determined by HPLC.

The following results are obtained:
RC (NBB)=99%
YC (HNBB)=82%.

### EXAMPLE 11

3.94 g of chlorobenzene (35 mmol, 7 eq.), 1.05 g of iron chloride (6.5 mmol, 1.3 eq.) and 0.72 g of parahydroxybenzoic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 4 hours. The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure (about 12 mm of mercury).

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

1.38 g of 2-n-butyl-5-nitro-8-(4′-hydroxy)acetophenonebenzofuran (4.08 mmol, which corresponds to a yield of 81.6%) are then obtained in the form of a light-grey solid.

The NMR characteristics of the resulting product are as follows:

¹H NMR (200 MHz, CDCl₃): δ (ppm)=0.87-0.93 (t, 3H, J=7.2 Hz); 1.30-1.42 (m, 2H, J=7 Hz); 1.72-1.83 (m, 2H, J=7.6 Hz); 2.91-2.96 (t, 2H, J=7.6 Hz); 6.95-6.98 (d, 2H, J=8.3 Hz); 7.56-7.59 (d, 1H, J=9 Hz); 7.79-7.82 (d, 2H, J=8.7 Hz); 8.21-8.25 (dd, 1H, J=9.3 Hz); 8.34 (d, 1H, J=2.1 Hz).

### EXAMPLE 12

3.94 g of chlorobenzene (35 mmol, 7 eq.), 1.05 g of iron chloride (6.5 mmol, 1.3 eq.) and 0.64 g of benzoic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 4 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 in the course of the elution.

1.31 g of 2-n-butyl-5-nitro-8-acetophenonebenzofuran (4.06 mmol, which corresponds to a yield of 81.3%) in the form of orange crystals (m.p.=55.1° C.) are then obtained.

The NMR characteristics of the resulting product are as follows:

¹H NMR (200 MHz, CDCl₃): δ (ppm)=0.79-0.84 (t, 3H, J=7.6 Hz); 1.26-1.31 (m, 2H); 1.64-1.72 (m, 2H); 2.80-2.85 (t, 2H, J=7.3 Hz); 7.43-7.52 (m, 3H); 7.57-7.62 (m, 1H); 7.73-7.76 (d, 2H, J=8.3 Hz); 8.13-8.17 (dd, 1H, J₁=8.9 Hz, J₂=2.4 Hz); 8.27-8.28 (d, 1H, J=2.2 Hz).

### EXAMPLE 13

3.94 g of chlorobenzene (35 mmol, 7 eq.), 1.05 g of iron chloride (6.5 mmol, 1.3 eq.) and 0.80 g of para-anisic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 4 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

1.28 g of 2-n-butyl-5-nitro-8-(4′-methoxy)acetophenonebenzofuran (3.64 mmol, which corresponds to a yield of 72.7%) in the form of a white solid (m.p.=94.7° C.) are then obtained.

The NMR characteristics of the resulting product are as follows:

¹H NMR (200 MHz, CDCl₃): δ (ppm)=0.87-0.92 (t, 3H, J=7.2 Hz); 1.30-1.42 (m, 2H); 1.72-1.82 (m, 2H); 2.90-2.95 (t, 2H, J=7.9 Hz); 3.13 (s, 3H); 6.98-7.01 (d, 2H, J=9.3 Hz); 7.55-7.58 (d, 1H, J=9.6 Hz); 7.82-7.85 (d, 2H, J=9 Hz); 8.20-8.24 (dd, 1H, J₂=9.2 Hz, J₂=2.4 Hz); 8.33-8.34 (d, 1H, J=2.7 Hz).

### EXAMPLE 14

3.94 g of chlorobenzene (35 mmol, 7 eq.), 1.05 g of iron chloride (6.5 mmol, 1.3 eq.) and 0.71 g of para-toluenic acid (5.25 mmol, 1.05 eq.) are introduced into a 30 ml Schott tube with magnetic stirring at 500 rpm and at ambient temperature. 1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 5 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

1.40 g of 2-n-butyl-5-nitro-8-(4′-methyl)acetophenonebenzofuran (4.17 mmol, which corresponds to a yield of 83.4%) are then obtained in the form of light-yellow crystals (m.p.=66.9° C.).

The NMR characteristics of the resulting product are as follows:

¹H NMR (200 MHz, CDCl₃): δ (ppm)=0.78-0.83 (t, 3H, J=7.6 Hz); 1.20-1.32 (m, 2H); 1.63-1.73 (m, 2H); 2.39 (s, 3H); 2.79-2.84 (t, 2H, J=7.6 Hz); 7.22-7.25 (d, 2H, J=7.9 Hz); 7.47-7.50 (d, 1H, J=9.2 Hz); 7.64-7.66 (d, 2H, J=8.2 Hz); 8.10-8.14 (dd, 1H, J₁=9.2 Hz, J₂=2.4 Hz); 8.26-8.27 (d, 1H, J=2.1 Hz).

### EXAMPLE 15

3.94 g of chlorobenzene (35 mmol, 7 eq.), 1.05 g of iron chloride (6.5 mmol, 1.3 eq.) and 0.82 g of 4-chlorobenzoic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 5 days.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

1.28 g of 2-n-butyl-5-nitro-8-(4′-chloro)-acetophenonebenzofuran (3.59 mmol, which corresponds to a yield of 71.7%) in the form of light-yellow crystals (m.p.=64° C.) are then obtained.

The NMR characteristics of the resulting product are as follows:

¹H NMR (200 MHz, CDCl₃): δ (ppm)=0.81-0.85 (t, 3H, J=7 Hz); 1.22-1.34 (m, 2H); 1.65-1.75 (m, 2H); 2.80-2.86 (t, 2H, J=7.9 Hz); 7.43-7.46 (dd, 2H, J₁=6.7 Hz, J₂=1.8 Hz); 7.50-7.53 (d, 1H, J=8.4 Hz); 7.70-7.72 (dd, 2H, J₁=6.4 Hz, J₂=1.8 Hz); 8.15-8.19 (dd, 1H, J₁=9.2 Hz, J₂=2.4 Hz); 8.28 (d, 1H, J=2.4 Hz).

### EXAMPLE 16

1.97 g of chlorobenzene (17.5 mmol, 7 eq.), 0.53 g of iron chloride (3.25 mmol, 1.3 eq.) and 0.5 g of trifluoro-para-toluenic acid (2.63 mmol, 1.05 eq.) are introduced into a 30 ml Schott tube with magnetic stirring at 500 rpm and at ambient temperature.

0.55 g of 2-n-butyl-5-nitrobenzofuran (2.5 mmol, 1 eq.) and 0.56 g of trichloromethylsilane (3.75 mmol, 1.5 eq.) are then added dropwise, and then the reaction mixture is put, with stirring, at a temperature of 40° C. for 24 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 in the course of the elution.

0.35 g of 2-n-butyl-5-nitro-8-(4′-trifluoromethyl)-acetophenonebenzofuran (0.86 mmol, which corresponds to a yield of 34.5%) is then obtained in the form of a yellow oil.

The NMR characteristics of the resulting product are as follows:

¹H NMR (200 MHz, CDCl₃): δ (ppm)=0.79-0.84 (t, 3H, J=7.6 Hz); 1.23-1.31 (m, 2H); 1.64-1.74 (m, 2H); 2.77-2.82 (t, 2H, J=7.3 Hz); 7.52-7.55 (d, 1H, J=8.5 Hz); 7.73-7.75 (d, 2H, J=8 Hz); 7.84-7.87 (d, 2H, J=8 Hz); 8.16-8.20 (dd, 1H, J₁=9.2 Hz, J₂=2.4 Hz); 8.29-8.30 (d, 1H, J=2.1 Hz).

### EXAMPLE 17

3.94 g of chlorobenzene (35 mmol, 7 eq.), 1.05 g of iron chloride (6.5 mmol, 1.3 eq.) and 0.80 g of meta-anisic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 5 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

1.02 g of 2-n-butyl-5-nitro-8-(3′-methoxy)-acetophenonebenzofuran (2.89 mmol, which corresponds to a yield of 57.6%) are then obtained in the form of yellow crystals (m.p.=67.5° C.).

The NMR characteristics of the resulting product are as follows:

¹H NMR (200 MHz, CDCl₃): δ (ppm)=0.80-0.85 (t, 3H, J=7.3 Hz); 1.22-1.34 (m, 2H); 1.65-1.75 (m, 2H); 2.81-2.86 (t, 2H, J=7.9 Hz); 3.81 (s, 3H); 7.11-7.15 (m, 1H); 7.26-7.37 (m, 3H); 7.49-7.52 (d, 2H, J=9.2 Hz); 8.14-8.17 (dd, 1H, J₁=8.9 Hz, J₂=2.4 Hz); 8.30-8.31 (d, 1H, J=2.4 Hz).

### EXAMPLE 18

3.94 g of chlorobenzene (35 mmol, 7 eq.), 1.05 g of iron chloride (6.5 mmol, 1.3 eq.) and 0.80 g of ortho-anisic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 22 hours. The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 in the course of the elution.

1.48 g of 2-n-butyl-5-nitro-8-(2′-methoxy)-acetophenonebenzofuran (4.19 mmol, which corresponds to a yield of 83.7%) are then obtained in the form of an orange oil.

The NMR characteristics of the resulting product are as follows:

¹H NMR (200 MHz, CDCl₃): δ (ppm)=0.78-0.83 (t, 3H, J=7.4 Hz); 1.17-1.29 (m, 2H); 1.58-1.68 (m, 2H); 2.74-2.77 (t, 2H, J=7.6 Hz); 3.62 (s, 3H); 6.95-6.97 (d, 1H, J=8.2 Hz); 7.01-7.06 (m, 1H); 7.34-7.37 (dd, 1H, J₁=7.6 Hz, J₂=1.8 Hz); 7.44-7.47 (d, 1H, J=8.2 Hz); 7.46-7.51 (m, 1H); 8.10-8.14 (dd, 1H, J₁=9.2 Hz, J₂=2.4 Hz); 8.30-8.31 (d, 1H, J=2.4 Hz).

### EXAMPLE 19

3.94 g of chlorobenzene (35 mmol, 7 eq.), 1.05 g of iron chloride (6.5 mmol, 1.3 eq.) and 0.32 g of acetic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 5 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 in the course of the elution.

There are then obtained 1.01 g of 2-n-butyl-5-nitro-benzofuranyl methyl ketone

(3.86 mmol, which corresponds to a yield of 77.1%) in the form of orange crystals (m.p.=52.2° C.).

The NMR characteristics of the resulting product are as follows:

¹H NMR (200 MHz, CDCl₃): δ (ppm)=0.90-0.95 (t, 3H, J=7.3 Hz); 1.36-1.44 (m, 2H); 1.70-1.80 (m, 2H); 2.63 (s, 3H); 3.11-3.16 (m, 2H); 7.46-7.49 (d, 1H, J=8.9 Hz); 8.16-8.20 (dd, 1H, J₁=9.2 Hz, J₂=2.4 Hz); 8.82-8.83 (d, 1H, J=2.1 Hz).

### EXAMPLE 20

3.94 g of chlorobenzene (35 mmol, 7 eq.), 1.05 g of iron chloride (6.5 mmol, 1.3 eq.) and 0.39 g of propionic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.), are then added dropwise and then the reaction mixture is put, with stirring, at a temperature of 40° C. for 5 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

1.16 g of 2-n-butyl-5-nitro-benzofuranyl ethyl ketone (4.21 mmol, which corresponds to a yield of 84.3%) are then obtained in the form of beige crystals (m.p. >200° C.).

The NMR characteristics of the resulting product are as follows:

¹H NMR (200 MHz, CDCl₃): δ (ppm)=0.89-0.94 (t, 3H, J=7.3 Hz); 1.21-1.25 (t, 3H, J=7 Hz); 1.34-1.46 (m, 2H); 1.69-1.79 (m, 2H); 2.90-2.99 (m, 2H); 3.12-3.17 (t, 2H, J=7.3 Hz); 7.466-7.495 (d, 1H, J=8.9 Hz); 8.16-8.19 (dd, 1H, J₁=9.2 Hz, J₂=2.4 Hz); 8.81 (s, 1H).

### EXAMPLE 21

3.94 g of chlorobenzene (35 mmol, 7 eq.), 1.05 g of iron chloride (6.5 mmol, 1.3 eq.) and 0.46 g of isobutyric acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 2 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 in the course of the elution.

1.22 g of 2-n-butyl-5-nitro-benzofuranyl isobutyryl ketone (4.20 mmol, which corresponds to a yield of 84%) are then obtained in the form of an orange oil.

The NMR characteristics of the resulting product are as follows:

¹H NMR (200 MHz, CDCl₃): δ (ppm)=0.88-0.93 (t, 3H, J=7.3 Hz); 1.20 (s, 3H); 1.23 (s, 3H); 1.32-1.44 (m, 2H); 1.68-1.78 (m, 2H); 3.08-3.13 (t, 2H, J=7.6 Hz); 3.24-3.33 (m, H); 7.50-7.47 (d, 1H, J=9.5 Hz); 8.14-8.18 (dd, 1H, J₁=9.2 Hz, J₂=2.4 Hz); 8.72-8.73 (d, 1H, J=2.5 Hz).

### EXAMPLE 22

3.94 g of chlorobenzene (35 mmol, 7 eq.), 1.05 g of iron chloride (6.5 mmol, 1.3 eq.) and 0.71 g of phenylacetic acid (5.25 mmol, 1.05 eq.) are introduced into a 30 ml Schott tube with magnetic stirring at 500 rpm and at ambient temperature.

1.09 g of 2-n-butyl-5-nitrobenzofuran (5 mmol, 1 eq.) and 1.12 g of trichloromethylsilane (7.5 mmol, 1.5 eq.) are then added dropwise, and then the reaction mixture is put, with stirring, at a temperature of 40° C. for 4 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

1.18 g of 2-n-butyl-5-nitro-benzofuranyl phenyl ketone (3.50 mmol, which corresponds to a yield of 69.9%) are then obtained in the form of white crystals (m.p.=86.8° C.).

The NMR characteristics of the resulting product are as follows:

¹H NMR (200 MHz, CDCl₃): δ (ppm)=0.87-0.92 (t, 3H, J=7.3 Hz); 1.31-1.43 (m, 2H); 1.66-1.77 (m, 2H); 3.11-3.16 (t, 2H, J=7.6 Hz); 4.26 (s, 2H); 7.20-7.31 (m, 5H); 7.48-7.51 (d, 1H, J=9.2 Hz); 8.17-8.20 (dd, 1H, J₁=8.9 Hz, J₂=2.4 Hz); 8.87 (d, 1H, J=2.1 Hz).

### EXAMPLE 23

3.94 g of chlorobenzene (35 mmol, 7 eq.), 1.05 g of iron chloride (6.5 mmol, 1.3 eq.) and 0.32 g of acetic acid (5.25 mmol, 1.05 eq.) are introduced into a 30 ml Schott tube with magnetic stirring at 500 rpm and at ambient temperature.

0.54 g of anisole (5 mmol, 1 eq.) and 1.12 g of trichloromethylsilane (7.5 mmol, 1.5 eq.) are then added dropwise, and then the reaction mixture is put, with stirring, at a temperature of 40° C. for 16 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

0.59 g of 4′-methoxyacetophenone (3.94 mmol, which corresponds to a yield of 78.7%) is then obtained in the form of an orange oil.

The NMR characteristics of the resulting product are as follows:

¹H NMR (200 MHz, CDCl₃): δ (ppm)=2.48 (s, 3H); 3.80 (s, 3H); 6.85-6.88 (d, 2H, J=9 Hz); 7.85-7.88 (d, 2H, J=9 Hz).

### EXAMPLE 24

3.94 g of chlorobenzene (35 mmol, 7 eq.), 1.05 g of iron chloride (6.5 mmol, 1.3 eq.) and 0.32 g of acetic acid (5.25 mmol, 1.05 eq.) are introduced into a 30 ml Schott tube with magnetic stirring at 500 rpm and at ambient temperature.

0.46 g of toluene (5 mmol, 1 eq.) and 1.12 g of trichloromethylsilane (7.5 mmol, 1.5 eq.) are then added dropwise, and then the reaction mixture is put, with stirring, at a temperature of 40° C. for 2.5 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

0.57 g of 4′-methylacetophenone (4.25 mmol, which corresponds to a yield of 85%) is then obtained in the form of an orange oil.

The NMR characteristics of the resulting product are as follows:

¹H NMR (200 MHz, CDCl₃): δ (ppm)=2.34 (s, 3H); 2.51 (s, 3H); 7.18-7.20 (d, 2H, J=7.2 Hz); 7.78-7.81 (d, 2H, J=8.1 Hz).

### EXAMPLE 25

3.94 g of chlorobenzene (35 mmol, 7 eq.), 1.05 g of iron chloride (6.5 mmol, 1.3 eq.) and 0.32 g of acetic acid (5.25 mmol, 1.05 eq.) are introduced into a 30 ml Schott tube with magnetic stirring at 500 rpm and at ambient temperature.

1.12 g of trichloromethylsilane (7.5 mmol, 1.5 eq.) are then added dropwise, and then the reaction mixture is put, with stirring, at a temperature of 40° C. for 23 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

0.55 g of 4′-chloroacetophenone (3.57 mmol, which corresponds to a yield of 68%) is then obtained in the form of an orange-brown oil.

The NMR characteristics of the resulting product are as follows:

¹H NMR (200 MHz, CDCl₃): δ (ppm)=2.52 (s, 3H); 7.34-7.39 (m, 2H); 7.80-7.85 (m, 2H).

### EXAMPLE 26

1.05 g of iron chloride (6.5 mmol, 1.3 eq.) and 0.32 g of acetic acid (5.25 mmol, 1.05 eq.) are introduced into a 30 ml Schott tube with magnetic stirring at 500 rpm and at ambient temperature.

2 g of meta-xylene (18.9 mmol, 3.8 eq.) and 1.12 g of trichloromethylsilane (7.5 mmol, 1.5 eq.) are then added dropwise, and then the reaction mixture is put, with stirring, at a temperature of 40° C. for 24 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

0.63 g of 2′,4′-dimethylacetophenone (4.23 mmol, which corresponds to a yield of 80.6%) is then obtained in the form of a brown oil.

The NMR characteristics of the resulting product are as follows:

¹H NMR (200 MHz, CDCl₃): δ (ppm)=2.30 (s, 3H); 2.47 (s, 3H); 2.50 (s, 3H); 7.00-7.02 (d, 2H, J=7.2 Hz); 7.56-7.59 (d, 1H, J=8.7 Hz).

### EXAMPLE 27

3.94 g of chlorobenzene (35 mmol, 7 eq.) and 1.05 g of iron chloride (6.5 mmol, 1.3 eq.) are introduced into a 30 ml Schott tube with magnetic stirring at 500 rpm and at ambient temperature.

0.76 g of para-anisic acid (5 mmol, 1 eq.) and 1.12 g of trichloromethylsilane (7.5 mmol, 1.5 eq.) are then added dropwise, and then the reaction mixture is put, with stirring, at a temperature of 40° C. for 24 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

0.41 g of 4′-chloro-4-methoxybenzophenone (1.67 mmol, which corresponds to a yield of 33.4%) is then obtained in the form of a brown oil.

The NMR characteristics of the resulting product are as follows:

¹H NMR (200 MHz, CDCl₃): δ (ppm)=3.84 (s, 3H); 6.90-6.93 (d, 2H, J=9.3 Hz); 7.38-7.41 (d, 2H, J=8.7 Hz); 7.64-7.66 (d, 2H, J=8.7 Hz); 7.72-7.75 (d, 2H, J=9 Hz).

### EXAMPLE 28

3.94 g of chlorobenzene (35 mmol, 7 eq.), 1.05 g of iron chloride (6.5 mmol, 1.3 eq.) and 0.75 g of 3-phenylpropionic acid (5 mmol, 1 eq.) are introduced into a 30 ml Schott tube with magnetic stirring at 500 rpm and at ambient temperature.

1.12 g of trichloromethylsilane (7.5 mmol, 1.5 eq.) are then added dropwise, and then the reaction mixture is put, with stirring, at a temperature of 40° C. for 18 hours.

The reaction mixture is then cooled to 0° C. by means of an ice-bath, and 3 ml of ethanol are added.

The mixture is diluted with 25 ml of chlorobenzene and filtered over sintered glass of no. 2 porosity.

The filtrate (organic phase) is then washed three times with 25 ml of a 1 N hydrochloric acid solution.

The organic phase is dried over magnesium sulfate and then concentrated under reduced pressure.

The product is isolated from the crude reaction mixture by separation on a column of silica (silica gel 60 of diameter 0.2 to 0.5 mm), the eluant being a heptane/ethyl acetate mixture going from a ratio of 95:5 to 80:20 during the elution.

0.59 g of 1-indanone (4.5 mmol, which corresponds to a yield of 90%) is then obtained in the form of white crystals.

The NMR characteristics of the resulting product are as follows:

¹H NMR (200 MHz, CDCl₃): δ (ppm)=2.58-2.62 (t, 2H, J=6 Hz); 3.04-3.08 (t, 2H, J=6 Hz); 7.28-7.31 (m, 1H); 7.38-7.41 (m, 1H); 7.48-7.50 (m, 1H); 7.66-7.69 (m, 1H).

### EXAMPLE A (COMPARATIVE)

280 mg of 4-hydroxybenzoic acid, 420 mg of 5-nitro-2-butylbenzofuran, 1.4 ml of phosphorus oxychloride and 600 mg of zinc(II) chloride are introduced successively at 23° C., under an inert atmosphere, into a 30 ml reactor equipped with a magnetic stirring system.

The mixture is then heated to 60° C., with stirring, over a period of 15 hours, and then cooled to 20° C.

5 ml of acetonitrile are then added, and stirring is carried out for a further 15 minutes.

The totality of the reaction mass is then weighed, and its composition is determined by HPLC.

The following results are obtained:
RC (NBB)<2%
AY (HNBB)<1%
RC (PHBA)=99%

1-35. (canceled)

36. A method for acylation of an aromatic compound, comprising reacting an aromatic compound and an acylating agent of the carboxylic acid type, in the presence of a Lewis acid and of a silylated reagent selected from the group consisting of halosilanes and halosiloxanes.

37. The method according to claim 36, wherein the aromatic compound has the following formula: 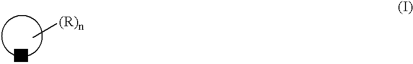
in which:
A represents the residue of a ring forming all or a part of a monocyclic or polycyclic, aromatic, carbocyclic or heterocyclic system,
each R represents a hydrogen atom or a substituent, and n represents the number of substituent(s) on the ring.

38. The method according to claim 37, wherein the aromatic compound has formula (I) in which the residue A represents the residue:
(1) of a monocyclic or polycyclic, aromatic carbocyclic compound,
(2) of a monocyclic or polycyclic, aromatic heterocyclic compound, or
(3) of a compound constituted by a sequence of rings, as defined in paragraphs (1) and/or (2), bonded together:
(a) by a valence bond,
(b) by a methylene, an isopropylidene or another alkylene or alkylidene group having up to 4 carbon atoms,
(c) by one of the following groups:
—O—, —CO—, —COO—, —OCOO—, —S—, —SO—, —SO₂—, —NR₀—, or —CO—NR₀—, in which formulae R₀ represents a hydrogen atom or an alkyl group having from 1 to 4 carbon atoms, a cyclohexyl group or a phenyl group.

39. The method according to claim 38, wherein the aromatic compound has formula (I) in which the residue A represents the residue of a compound of the benzene type or of the benzofuran type.

40. The method according to claim 37, wherein the aromatic compound has formula (I) in which the group or groups R, which are identical or different, represent(s) one of the following groups:
a hydrogen atom, methyl, ethyl, propyl, isopropyl, butyl, isobutyl, sec-butyl, tert-butyl, or another linear or branched alkyl group having up to 6 carbon atoms,
vinyl, allyl, or another linear or branched alkenyl group having up to 6 carbon atoms,
the groups methoxy, ethoxy, propoxy, isopropoxy, butoxy, an alkenyloxy group, an allyloxy group, a phenoxy group or another linear or branched alkoxy group having up to 6 carbon atoms,
a cyclohexyl, phenyl or benzyl group,
an acyl group having from 2 to 6 carbon atoms, or
a group of the formula:
—R₁—OH
—R₁—COOR₂
—R₁—CHO
—R¹—NO₂
—R₁—CN
—R₁—N(R₂)₂
—R₁—NH—P
—R₁—CO—N(R₂)₂
—R₁—NH—SO₂—R₃
—R¹—X
—R₁—CF₃
in which formulae
R₁ represents a valence bond, methylene, ethylene, propylene, isopropylene, isopropylidene or another saturated or unsaturated, linear or branched divalent hydrocarbon group having up to 6 carbon atoms,
the R₂ groups, which are identical or different, represent a hydrogen atom or a linear or branched alkyl group having from 1 to 6 carbon atoms,
R₃ represents:
a methyl group, an ethyl group, or another alkyl group having up to 0 carbon atoms;
an alkyl group having from 1 to 10 carbon atoms carrying a halogen atom,
a CF₃ group or an ammonium N(R₆)₄ group, wherein the R₆ groups are identical or different and each represent an alkyl group having from 1 to 4 carbon atoms,
a cyclohexyl group or another cycloalkyl group having from 3 to 8 carbon atoms,
a phenyl group, or another aryl group having from 6 to 12 carbon atoms, an aryl group having from 6 to 12 carbon atoms carrying a methyl or ethyl group or another alkyl group having up to 10 carbon atoms, a halogen atom, a CF₃ group or a NO₂ group,
a CX₃ group in which X represents a fluorine, chlorine or bromine atom, or
a Cₚ Hₐ F_{b} group in which p represents a number ranging from 1 to 10, b represents a number ranging from 3 to 21, and a+b=2 p+1,
P represents a protecting group for the amino group, and
X represents a chlorine, bromine or fluorine atom or other halogen atom.

41. The method according to claim 37, wherein the aromatic compound has formula (I) in which the aromatic ring carries a —R₁—COOR₂ group in which R₂ represents a hydrogen atom and R₁ represents an alkylene group having at least 1, 2 or 3 carbon atoms or in which n is greater than or equal to 2 and two R groups and the two successive atoms of the aromatic ring are bonded together by an alkylene, alkenylene or alkenylidene group having from 2 to 4 carbon atoms to form a saturated, unsaturated or aromatic heterocycle having from 5 to 7 carbon atoms.

42. The method according to claim 41, wherein the protecting group P is a group acetyl, benzoyl or another acyl group, or a group BOC (butyloxycarbonyl), Cbz (carbobenzoxy), Fmoc (9-fluorenylmethoxycarbonyl) or MSOC (methanesulfenyl-2-ethoxycarbonyl).

43. The method according to claim 36, wherein the aromatic compound has the formula (Ia): 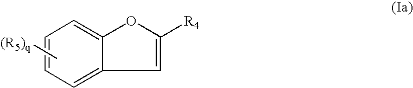
in which formula:
R₄ represents a phenyl group or a linear or branched alkyl group having from 1 to 12 carbon atoms, or a phenyl group substituted by an alkyl group having from 1 to 4 carbon atoms, or a halophenyl group,
R₅, each of which is identical or different, represents a hydrogen atom or a substituent, and
q is a number equal to 0, 1, 2 or 3.

44. The method according to claim 43, wherein the aromatic compound has formula (Ia) in which R₅ represents:
a nitro group,
methyl, ethyl, propyl, isopropyl, butyl, isobutyl, sec-butyl, tert-butyl or another linear or branched alkyl group having up to 6 carbon atoms,
an alkoxy group having from 1 to 6 carbon atoms,
a —N(R₂)₂ group, R₂ having the meaning given hereinbefore,
a —NH—P group, P having the meaning given hereinbefore,
a —CO—N(R₂)₂ group, R₂ having the meaning given hereinbefore,
a —NH—SO₂—R₃group, R₃ having the meaning given hereinbefore,
a halogen atom, or
a trifluoromethyl group.

45. The method according to claim 43, wherein the aromatic compound has formula (Ia) in which R₄ represents an alkyl group having from 1 to 4 carbon atoms.

46. The method according to claim 36, wherein the aromatic compound is 5-nitro-2-butylbenzofuran, anisole, toluene, monochlorobenzene, or xylenes.

47. The method according to claim 36, wherein the acylating agent has the following formula: 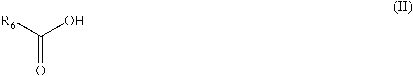
in which formula:
R₆ represents a hydrocarbon group having from 1 to 20 carbon atoms, a linear or branched, saturated or unsaturated, acyclic aliphatic group; a monocyclic or polycyclic, saturated, unsaturated, or aromatic, carbocyclic or heterocyclic group;
or a linear or branched, saturated or unsaturated aliphatic group carrying a cyclic substituent.

48. The method according to claim 47, wherein the acylating agent has formula (II) in which R₆ represents:
a saturated or unsaturated, linear or branched acyclic aliphatic group having from 1 to 12 carbon atoms, the hydrocarbon chain being interrupted or not by a carbon, a hetero-atom or a functional group and carries or not one or more substituents,
a linear or branched, saturated or unsaturated aliphatic group carrying a cyclic substituent,
a saturated or unsaturated carbocyclic or heterocyclic group having from 5 to 6 carbon atoms, or
a condensed or uncondensed, monocyclic or polycyclic, aromatic carbocyclic or heterocyclic group.

49. The method according to claim 47, wherein the acylating agent has formula (II) in which R₆ represents a ring carrying one or more R₇ groups which are:
methyl, ethyl, propyl, isopropyl, butyl; isobutyl, sec-butyl, tert-butyl or another linear or branched alkyl group having up to 6 carbon atoms,
vinyl, allyl or another linear or branched alkenyl group having up to 6 carbon atoms, methoxy, ethoxy, propoxy, isopropoxy, butoxy groups, a 2-oxypropionic group or another linear or branched alkoxy group having up to 6 carbon atoms,
a —OH group,
a —CHO group,
an acyl group having from 2 to 6 carbon atoms,
a —COOR₂ group, wherein R₂ has the meaning given hereinbefore,
a —NO₂ group,
a CO—N(R₂)₂ group, wherein R₂ has the meaning given hereinbefore,
a fluorine, chlorine, or bromine atom or other halogen atom, or
a —CF₃ group.

50. The method according to claim 47, wherein the acylating agent has the formula: 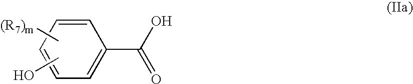
in which:
R₇, each of which is identical or different, represents a hydrogen atom or a
substituent,
m is a number less than 4.

51. The method according to claim 50, wherein the acylating agent has formula (IIa) in which the R₇ group represents a hydrogen atom, an alkyl or alkoxy group having from 1 to 4 carbon atoms, or a nitro group or a nitrile group.

52. The method according to claim 50, wherein the acylating agent has formula (IIa) in which m is equal to 0 or 1.

53. The method according to claim 47, wherein the acylating agent is acetic acid, propionic acid, isobutyric acid, lactic acid, trifluoroacetic acid, phenylacetic acid, 3-phenylpropionic acid, benzoic acid, 2-hydroxybenzoic acid, 3-hydroxybenzoic acid, 4-hydroxybenzoic acid, para-anisic acid, meta-anisic acid, ortho-anisic acid, para-toluenic acid, trifluoropara-toluenic acid, 4-chlorobenzoic acid, or other chlorobenzoic acid.

54. The method according to claim 36, wherein the Lewis acid is a rare earth or bismuth trifluoromethanesulfonate or another salt having an acetate, propionate, benzoate, methanesulfonate, trifluoromethanesulfonate or another organic counter-ion, of the metal or metalloid elements of groups (IIIa), (IVa), (VIII), (IIb), (IIIb), (IVb), (Vb) and (VIb) of the periodic system of elements.

55. The method according to claim 36, wherein the Lewis acid is a salt having a chloride, bromide, iodide, sulfate, oxide or another inorganic counter-ion, or analogous product of the metal or metalloid elements of groups (IIa), (IIIa), (IVa), (VIII), (IIb), (IIIb), (IVb), (Vb) and (Vlb) of the periodic system of elements.

56. The method according to claim 54, wherein the salt is a salt of magnesium or another element of group (IIa) of the periodic system, scandium, yttrium, lanthanides or another element of group (IIa); titanium or zirconium or another element of group (IVa); iron or another element of group (VIII); zinc or another element of group (IIb); boron, aluminum, gallium, indium or another element of group (IIIb); tin or another element of group (IVb); bismuth or another element of group (Vb); or tellurium or another element of group (Vlb).

57. The method according to claim 56, wherein the Lewis acid is magnesium chloride MgCl₂, zirconium chloride ZrCl₄, ferric chloride FeCl₃, zinc chloride ZnCl₂, aluminum chloride AlCl₃, aluminum bromide AlBr₃, gallium chloride GaCl₃, indium chloride InCl₃, stannic chloride SnCl₄, bismuth chloride BiCl₃, boron trifluoride BF₃, bismuth triflate or another metal halide.

58. The method according to claim 57, wherein the Lewis acid is ferric chloride.

59. The method according to claim 36, wherein the silylated reagent is a polyhalosilane or a polyhalosiloxane.

60. The method according to claim 59, wherein the silylated reagent is a halosilane having the following formula:
(R₈)_{z}—Si —X_{4-z} (IIIa)
in which
R₈ represents a hydrogen atom, or an alkyl, alkenyl, cycloalkyl, aryl or arylalkyl group,
R₈ represents at most one hydrogen atom,
X represents a chlorine, bromine or iodine atom, and
z is a number equal to 0, 1 or 2.

61. The method according to claim 60, wherein the halosilane has formula (IIIa) in which R₈ is a phenyl group, a methyl group or another alkyl group having up to 4 carbon atoms.

62. The method according to claim 60, wherein the halosilane is selected from the group consisting of Me₂SiCl₂, MeSiCl₃, SiCl₄, and MeSiHCl₂.

63. The method according to claim 59, wherein the silylated reagent is a halosiloxane having the following formula: 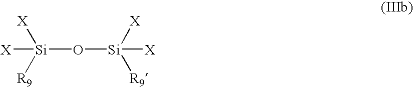
in which:
R₉ and R₉′, which are identical or different, each represent an alkyl, alkenyl, cycloalkyl, aryl or arylalkyl group, and
X represents a chlorine, bromine or iodine atom.

64. The method according to claim 63, wherein the halosiloxane is selected from the group consisting of 1,1,3,3-tetrachloro-1,3-dimethylsiloxane, 1,1,3,3-tetrachloro-1,3-diethylsiloxane, 1,1,3,3-tetrachloro-1,3-diisopropylsiloxane, and 1,1,3,3-tetrachloro-1,3-divinylsiloxane.

65. The method according to claim 36, wherein the reaction is conducted in the presence of monochlorobenzene or another halogenated or non-halogenated aliphatic or aromatic hydrocarbon solvent or other organic solvent.

66. The method according to claim 36, wherein the ratio between the number of moles of acylating agent and the number of moles of aromatic compound varies between 0.5 and 1.5.

67. The method according to claim 36, wherein the amount of Lewis acid, expressed by the ratio between the number of moles of catalyst and the number of moles of compound of formula (I), varies between 0.01 and 2.

68. The method according to claim 36, wherein the ratio between the number of halogen atoms of the silylated reagent and the number of moles of compound of formula (II) varies between 1 and 10.

69. The method according to claim 36, wherein the reaction temperature lies between 0° C. and 160° C.

70. The method according to claim 36, wherein the reaction is conducted at atmospheric pressure and under a controlled atmosphere of nitrogen or another inert gases.
